(19) United States
(12) Patent Application Publication
(10) Pub. No.: US 2018/0243247 A1
(43) Pub. Date: Aug. 30, 2018

(54) DRUG COMBINATIONS FOR CEREBROVASCULAR DISEASE

(71) Applicant: Bryan J. Katz, Baltimore, MD (US)

(72) Inventor: Bryan J. Katz, Baltimore, MD (US)

(21) Appl. No.: 15/907,239

(22) Filed: Feb. 27, 2018

Related U.S. Application Data
(60) Provisional application No. 62/464,985, filed on Feb. 28, 2017.

(51) Int. Cl. A61K 31/19; A61K 31/135; A61K 31/436; A61K 31/4422; A61K 31/165; A61P 25/28

(52) U.S. Cl. CPC A61K 31/19; A61K 31/135; A61P 25/28; A61K 31/4422; A61K 31/165; A61K 31/436

(57) ABSTRACT

The invention provides a drug combination of at least two compounds selected from the group consisting of Valproate, Ketamine, Tacrolimus, Clevidipine and Joro Spider Toxin that can be administered parenterally or orally to patients over 18 years of age at the onset of stroke symptoms or Transient Ischemic Attack (TIA). The drug combination may be administered regardless of stroke etiology (ischemic vs. hemorrhagic); may be given up to 6 hours following the onset of stroke and should not interfere with the subsequent action of thrombolytic agents such as tPA (Tissue Plasminogen Activator).

This application claims the priority benefit under 35 U.S.C. section 119 of U.S. Provisional Patent Application No. 62/464,985 entitled “Drug Combination For Hemorrhagic Stroke Patients” filed on Feb. 28, 2017; and which is in its entirety herein incorporated by reference.

### FIELD OF THE INVENTION

The present invention relates to the field of medicine. It provides new compositions and methods for treating or protecting individuals in need thereof from cerebral ischemia or hypoxia. The invention also relates to new compositions and methods for treating stroke. More specifically, the invention relates to novel methods and drug combinations to protect neuronal cells from ischemia- or hypoxia-induced cell death. The compositions and methods of the invention may be used to treat brain ischemic or hypoxic injuries in any mammalian subject.

### BACKGROUND OF THE INVENTION

Stroke is a major cause of death and disability. Primary stroke prevention focuses on lifestyle modifications of risk factors while secondary stroke prevention aims to reduce the overall risk of recurrence in persons who have had a stroke.

Ischemia is a condition in which there is insufficient blood flow to a given part of the body to meet metabolic demand. This shortage of oxygen, glucose and other nutrients leads to tissue damage at the ischemic area. It can affect an entire organ, a limb or just part of a tissue, depending upon the vascular system involved. There are various types of ischemia with specific mechanisms, depending on the area experiencing the ischemic insult, but they all share overall processes responsible for such an insult with globally common consequences.

Many events can lead to an insufficient blood supply to a given tissue: atherosclerosis, thromboembolism, hypoglycemia, tachycardia, hypotension, outside compression of a blood vessel (e.g. by a tumor or following a trauma), embolism, sickle cell disease, localized extreme cold, tourniquet application, arteriovenous malformations, peripheral artery occlusive disease, hemorrhage.

One major consequence of ischemia is the lack of oxygen normally supplied through binding to hemoglobin in red blood cells. The affected tissue rapidly becomes hypoxic if not anoxic. This, added to the lack of glucose, the energy supply, leads to the release of proteolytic enzymes, reactive oxygen species and inflammatory mediators. This so-called ischemic cascade will ultimately cause cell death and tissue damage. Ischemia can thus develop in any part of the body, such as a limb, intestine, heart or brain. The heart and brain are among the organs that are the most quickly damaged by ischemia: cell necrosis ensues after only 3-4 minutes after onset.

Cerebral ischemia of the brain tissues results in brain cell damage and death. Unlike other tissues, which can survive extended periods of hypoxia, brain tissue is particularly sensitive to deprivation of oxygen or energy. Permanent damage to neurons can occur even during brief periods of hypoxia or ischemia. At present, there is no effective neuroprotective strategy for the treatment of cerebral ischemia or hypoxia. Cerebrovascular disease is the third most common cause of death worldwide (WHO 2008), being responsible for 10.8% of worldwide deaths. In addition, it is one of the first causes of long-term disability in Western countries, with more than 50% of patients being left with a motor disability and a significant loss of quality-adjusted life years (QALY). The risk of cerebral ischemia increasing with age; the burden of cerebral ischemia is becoming greater as the population is aging. The improvement of health care by the development of faster and more effective therapy would therefore have an important medical and socioeconomic impact worldwide and is greatly needed.

Symptoms of cerebral ischemia and their severity vary greatly depending on the cerebral region(s) affected. For instance, they may include weakness on one side of the body, impairments in speech or vision and/or mental confusion. Focal cerebral ischemia, which occurs when a blood clot has occluded a cerebral vessel and is confined to a specific region of the brain, is usually caused by thrombosis or embolism. It can also be caused by a lacunar infarct, in which a small arteriole (usually in the basal ganglia) ruptures. Patients with long standing diabetes are especially prone to lacunar infarcts. Global cerebral ischemia, which occurs when blood flow to the entire brain is stopped or drastically reduced, is commonly caused by cardiovascular disease. The area(s) of brain tissue affected as well as the delay in diagnosis and treatment are essential factors determining the outcome of cerebral ischemia, i.e., survival and level of disability after recovery.

One major consequence of cerebral ischemia is neuronal damage, which is mediated by the ischemic cascade that results in tissue damage leading to subsequent neuronal death and to disruption of the blood-brain barrier. It is estimated that 2 million brain cells die every minute after ischemic stroke onset. In addition, restoration of blood flow after a period of ischemia can actually be more damaging than the ischemia itself. The so-called reperfusion injury can result in acceleration of neuronal death.

There is, currently, no effective drug therapy to help patients during the acute phase of brain ischemia except thrombolysis and new endovascular devices or surgical techniques which will benefit a limited number of patients. This is due to one major issue: a very narrow therapeutic window of less than 6 h from ischemia onset. Another issue is the invasiveness and complexity of these procedures.

New therapies are currently contemplated which aim at (i) regenerating damaged brain areas to regain neurological function and (ii) targeting the ischemic cascade to minimize and even prevent brain damage. Unfortunately, so far, these new therapies have been effective in experimental settings but have failed to translate into clinical practice.

Stimulation of neurogenesis using endogenous repair mechanisms such as neuronal progenitor cells or transplantation of stem cells is being actively investigated. Unfortunately, issues such as survival of the cells, proper differentiation and proper connectivity of the new neuronal cells remain unsolved.

Antioxidant enzymes, primarily superoxide dismutase (SOD), in association with catalase, and glutathione peroxidase, have been tested in vivo but showed no improvement in cerebral blood flow or neurological recovery.

Newer therapeutic approaches with different modes of action and a wider therapeutic window are currently being investigated for ischemic stroke: glutamate antagonists, anti-inflammatory agents, anti-apoptotic agents, and ion-channel modulators.

Glutamate is the most abundant excitatory neurotransmitter in the mammalian nervous system. It activates glutamate receptors that are classified into three ionotropic classes (NMDA, AMPA and kainate receptors) and three metabotropic classes. Under normal conditions, glutamate concentration is maintained by glial and neuronal systems. During ischemia, an abnormally high concentration of extracellular glutamate is observed in the brain. Excessive accumulation of glutamate in synaptic clefts leads to the overactivation of glutamate receptors that results in pathological processes and finally in neuronal cell death. This process, termed excitotoxicity, is commonly observed in neuronal tissues under ischemic conditions. Glutamate receptor overactivation results in an accumulation of several ion species, especially calcium, within postsynaptic cells. Calcium overload is a key process of excitotoxicity. It results in deleterious cellular processes especially when specific structures organelles such as mitochondria or the endoplasmic reticulum are no longer able to sequester cytoplasmic calcium. Excessive calcium overload in mitochondria is associated with the increased generation of reactive oxygen species as well as the release of pro-apoptotic mitochondrial proteins (Cytochrome C), which are both detrimental to cell survival. Transient or sustained calcium influx into cells also activates a number of deleterious enzymes, including nitric oxide synthase, phospholipases, endonucleases, and proteases such as caspases and calpain.

Several glutamate receptor antagonists have been tested to counteract excitotoxicity. However, the effects of glutamate receptor antagonists, such as the NMDA receptor antagonists Selfotel, Eliprodil and Aptiganel (Cerestat), could not be validated in clinical studies, and several studies have been stopped. Dizolcipine (MK801), another NMDA receptor antagonist, is associated with numerous adverse effects. Calcium channel blockers such as Nimodipine and Flunarizine also showed no significant benefit when used as monotherapy versus placebo in clinical trials. A phase III trial with the AMPA receptor antagonist YM872 (zonampanel) is ongoing and seeks to determine its potential efficacy in combination with tPA thrombolysis.

A variety of anti-inflammatory drugs have been shown to reduce ischemic damage in animal studies. Commonly used anti-inflammatory agents are aspirin and the lipid-lowering statins. In addition, two leukocyte adhesion inhibitors, Enlimomab and LeukArrest, were studied in patients with ischemic stroke, but secondary effects seemed to be greater than their intended therapeutic effects.

Ion channel modulators (i.e., Nimodipine, Fosphenytoin, Maxipost) failed at phase III due to a lack of demonstrated benefit.

Anti-apoptotic agents such as caspase inhibitors have been shown to reduce the area of ischemic damage in rodent stroke models. Caspase inhibitors have not yet been tested in humans; as such their benefit remains uncertain.

Additionally, CNS neurons are totally dependent on aerobic respiration for ATP production. Ischemia, defined as an interruption of blood flow, is the most common etiology of CNS hypoxia in humans. A disruption in blood flow for as little as 10 seconds can lead to harmful (albeit reversible) metabolic alterations in CNS cells. Ischemia lasting for minutes, as in the case of a transient ischemic attack (TIA), produces a hypoxic insult to the brain that is largely reversible assuming blood flow is restored. (By clinical definition, TIA symptoms resolve in 24 hours or less). Longer periods of hypoxia invariably result in brain tissue infarction.

In humans this manifests as CVA (cerebrovascular accident) a.k.a. stroke, one of the five leading causes of mortality in the United States today. Over 800,000 Americans suffer a stroke annually; the one year mortality following a stroke is approximately 25%.¹ The majority of strokes (˜85%) are thromboembolic in nature; the remainder include hemorrhagic strokes, intracranial hemorrhage due to head trauma, and prolonged vasospasm. The pathogenesis of neuronal cell death following hypoxic insult can be thought of as a “three strikes” scenario:

a) Strike One: Glutamate excitotoxicity—the inability of hypoxic neurons to maintain ionic gradients leads to membrane depolarization and excessive release of glutamate at glutamatergic synapses. This effect is exacerbated by the impaired ability of hypoxic astrocytes to remove glutamate from the extracellular fluid.
b) Strike Two: Excessive glutamate binds to ionotropic receptors, including N-methyl D-aspartate (NMDA) receptors as well as α-amino-3-hydroxy-5-methyl-4-isoazolepropionic acid (AMPA) receptors, both of which allow uncontrolled calcium influx into neurons and glial cells.
c) Strike Three: The many toxic effects of elevated cytosolic calcium include the persistent activation of the phosphatase calcineurin; its subsequent dephosphorylation of nitric oxide synthetase (NOS); uncontrolled production of nitric oxide (NO) and other free radicals; damage to the mitochondria and nuclear DNA; and ultimately, the activation of apoptotic pathways culminating in cell death.

Clinically, stroke patients can receive thrombolytics, especially tissue plasminogen activator (tPA), during the 180 minutes following the onset of stroke symptoms. (Although 180 minutes is the optimal time window, some stroke centers administer tPA up to 6 hours after the onset of stroke). A crucially important consideration is that thrombolytics are indicated exclusively for the treatment of thromboembolic strokes. They are contraindicated in hemorrhagic strokes or in patients with bleeding disorders or who are on chronic anticoagulant therapy. In order to distinguish a thromboembolic stroke from a hemorrhagic one, a head CT must be performed, further delaying the initiation of thrombolytic therapy.

Due to the above-mentioned societal impact of cerebral ischemia, there is still a need for drugs effective in protecting neuronal cells during an ischemic or hypoxic event, following its onset, or in a preventive way in a patient at risk.

### BRIEF DESCRIPTION OF THE FIGURES

FIG. 1 shows a first embodiment of the invention illustrating how to prepare a dissolved composition for IV administration.

FIG. 2 illustrates another embodiment of preparing the dissolved compositions for IV administration.

### SUMMARY OF THE INVENTION

The present invention provides novel compositions and methods for treating brain ischemia and hypoxia. More specifically, the present invention stems from the discovery of specific drug combinations that provide remarkable neuroprotective effects against ischemia or hypoxia and therefore represent novel treatments for such conditions.

The invention thus discloses novel therapies for the treatment of brain ischemic or hypoxic injuries caused by various pathological conditions resulting in reduced blood flow or in reduced oxygen content.

The invention provides a drug combination of at least two compounds selected from the group consisting of Valproate, Ketamine, Tacrolimus, Clevidipine and Joro Spider Toxin that can be administered parenterally or orally to patients over 18 years of age at the onset of stroke symptoms or Transient Ischemic Attack (TIA). The drug combination may be administered regardless of stroke etiology (ischemic vs. hemorrhagic); may be given up to 6 hours following the onset of stroke and should not interfere with the subsequent action of thrombolytic agents such as tPA (Tissue Plasminogen Activator).

Another object of the invention relates more specifically to a composition comprising Valproate, Ketamine, Tacrolimus, Clevidipine and optionally Joro Spider Toxin, or any of their pharmaceutically acceptable salts, enantiomers, racemates, prodrugs, derivatives or metabolites, for use in the treatment of a brain ischemic or hypoxic injury.

A further object of the invention relates more specifically to a composition comprising Valproate, Ketamine, Tacrolimus, Clevidipine and Joro Spider Toxin, or any of their pharmaceutically acceptable salts, enantiomers, racemates, prodrugs, derivatives or metabolites, for use in the treatment of a brain ischemic or hypoxic injury.

The treatment may be preventive, particularly in subjects at risk, or curative, and may be used to treat focal or global ischemic injuries, as well as hypoxic injuries, of different origins. It is particularly suited to treat ischemia or hypoxia in subjects suffering from, having suffered from, or susceptible to suffer from, e.g., stroke, acute ischemic stroke, transient ischemic attack, acute hemorrhagic stroke, head trauma, brain hemorrhages, cardiac arrest, cerebral edema, hydrocephalus, asphyxia, thrombosis, embolism, thromboembolism, atherosclerosis, prolonged severe hypotension, intrauterine hypoxia, birth hypoxia, cardiac surgery complications or neurosurgery complications.

Another object of the invention relates to a composition as described above, for use to reduce, prevent or retard ischemia- or hypoxia-induced neuronal damage or neuronal cell death.

Another object of the invention relates to a composition as described above, for use to protect a subject from brain ischemia or hypoxia.

The present invention also relates to a composition comprising Valproate, Ketamine, Tacrolimus, Clevidipine and Joro Spider Toxin or any of their pharmaceutically acceptable salts, enantiomers, racemates, prodrugs, derivatives or metabolites, for use in the curative or prophylactic treatment of stroke in a subject.

The invention also relates to a composition as described above, for use to reduce, prevent or retard stroke-induced neuronal damage or neuronal cell death.

The invention also relates to pharmaceutical compositions per se comprising at least one drug combination as disclosed above. The pharmaceutical compositions of the invention typically comprise one or several pharmaceutically acceptable excipient(s) or carrier(s). As will be further disclosed in the present application, the compounds in a combinatorial therapy according to the invention may be formulated or administered to the subject together, separately or sequentially, possibly through different routes and protocols. In a preferred embodiment, the compositions of the invention are administered repeatedly to the subject.

The invention also relates to methods of treating brain ischemia or hypoxia in a subject in need thereof, the method comprising simultaneously, separately or sequentially administering to said subject a drug combination or a composition as disclosed above.

The present invention also relates to methods of treating stroke in a subject in need thereof, the method comprising simultaneously, separately or sequentially administering to said subject a drug combination or a composition as disclosed above.

A further object of this invention relates to the use of the above-described drug combinations or compositions for the manufacture of a medicament for the treatment of brain ischemia or hypoxia.

A further object of this invention relates to the use of the above-described drug combinations or compositions for the manufacture of a medicament for the treatment of stroke.

The treatment according to the invention may be used alone or in combination or in alternation with other therapies of such conditions. The invention is applicable to any mammalian subject, particularly humans.

The invention provides a properly designed drug cocktail that provides distinct advantages over the current approach to acute stroke management in the following ways: It would a) extend the time window for administering thrombolytics; b) have no significant contraindications; c) provide neuroprotection in any stroke regardless of etiology; and d) combat each of the above mentioned threats to cell viability. The drug cocktail in the invention contains at least two drugs selected from the group consisting of:

A1—Ketamine which is the only parenteral NMDA receptor antagonist already in clinical use. Drugs in this category would shield neurons from excess glutamate, potentially limiting its excitotoxic effects.

A2—Tacrolimus/FK506—This immunosuppressant drug exhibits neuroprotective effects in rodent models of CNS ischemia.² The benefits of FK506 may involve multiple mechanisms. First, a complex of FK506 and an immunophillin binding protein inhibits the cytosolic phosphatase calcineurin, thereby decreasing the dephosphorylation of iNOS (an isoform of the enzyme known as inducible NOS), which then minimizes production of NO, albeit in macrophages more so than in neurons or glial cells. Second, FK506 is known to inhibit IL-2 gene expression. This decreases T-cell infiltration into the hypoxic brain region, which in turn limits CNS inflammation and edema. Third, FK506, probably in conjunction with FKBP12, induces de novo RNA synthesis in astrocytes within two hours of incubation. The identities of the target genes are unknown but may involve the upregulation of anti-apoptotic genes such as bcl-2 and bcl-XL.³ Another study suggests the drug may down-regulate the expression of inflammatory cytokine genes including IL-1, IL-6, and TNFα.

A3—Calcium channel blockers especially Clevidipine should help counter the toxic effects of extracellular calcium influx. Although many CCBs are used clinically as anti-arrhythmic and antihypertensive agents, none is currently indicated for treatment of acute CVA. Nimodipine is used to relieve vasospasm secondary to subarachnoid hemorrhage; however, it cannot be given parenterally. Of all the peripheral dihydropyridine CCBs, only nicardipine and clevidipine can be injected safely by IV.

As formulated currently, Clevidipine comes as an egg/oil emulsion 0.5 mg/mL. All sources advise that Clevidipine should be administered separately in its own IV bag/tubing at 1-2 mg/hr then increased to 15 mg/hr.

However, if future formulations of Clevidipine are available as dry powder that can be diluted to a 0.5 mg/mL final dose, then a dry powder mixture containing Clevidipine is feasible and should be included in this patent.

### For IV Formulations, Nicardipine can be Substituted for Clevidipine.

IV Nicardipine is available as pre-mixed Cardene 20 mg or 40 mg or a generic equivalent. In situations where Clevidipine is unavailable (or does not mix uniformly with other agents), another IV dihydropyridine calcium channel blocker can be used—namely Nicardipine 0.1 mg/mL. Nicardipine is given 15 mg/hr IV. Diluting 30 mg in 300 mL IV fluid would work. The other drugs should still dissolve in 300 mL of diluent instead of 500 mL.

Other CCBs

Nimodipine is available only in oral form in the USA. However IV Nimodipine is available outside the U.S.

Nifedipine (immediate release or extended release) should only be used orally. Other oral dihydropyridine CCBs include Amlodipine, Felodipine, Isradipine and Nisoldipine.

Diltiazem and Verapamil are CCBs but belong to different drug classes. Verapamil is a phenylalkylamine and Diltiazem is a benzothiazepine. Both act primarily on the heart to slow AV node conduction, not the periphery or CNS. Diltiazem can be given IV but would lower heart rate; this effect is usually not desirable during a stroke.

A4—Valproate (GABA-mimetic). Anecdotal evidence suggests that FK506 can reduce the seizure threshold, especially in the setting of CNS hypoxia. Valproate's structure resembles the inhibitory neurotransmitter gamma amino butyric acid (GABA). Adding an effective anticonvulsant would be sensible. Furthermore, the GABA agonist effects of A4 (hyperpolarizing the cell by increasing chloride ion influx) may be beneficial in blunting glutamate excitotoxicity.

A5—Joro Spider Toxin (JSTX-3)—AMPA receptor antagonists, which block a specific class of glutamate gated ion channel on the postsynaptic membrane, have yet to be tested in human trials. One of these agents, a polyamine known as JSTX-3 (a component of Joro spider venom from certain orb weaving spiders native to Japan and Korea) dramatically reduces calcium influx through the AMPA receptor. The lack of an effective AMPA receptor blocker may account for the difficulty in minimizing glutamate excitotoxicity using NMDA receptor antagonists or CCBs alone.

### DETAILED DESCRIPTION OF THE INVENTION

The present invention provides new therapeutic approaches for treating brain ischemic or hypoxic injuries. The invention discloses specific drug combinations and methods allowing effective protection of neuronal cells against ischemia or hypoxia.

Within the context of the present invention, the terms brain “ischemia” or “ischemic injury” refer to a condition occurring in subjects having insufficient blood supply to the brain or a region of the brain. More specifically, the term ischemia or ischemic injury refers to any pathological state of the brain or a region thereof in which cerebral blood flow is insufficient to meet the metabolic demands of cerebral tissue, resulting in damage to the entire brain or wide areas of the brain (global ischemia) or to a specific region of the brain (focal ischemia).

The terms brain “hypoxia” or “hypoxic injury” refer to a condition occurring in subjects having reduced oxygen supply to the brain or a region of the brain. Hypoxia usually results from ischemic events. However, hypoxia may also occur without ischemia, when the oxygen content in blood vessels decreases without diminution of blood flow. Hypoxia according to the invention also comprises anoxia which is a complete tissue deprivation of oxygen supply.

The term “brain ischemic” or “brain hypoxic injury” therefore particularly includes a brain injury in which neuronal cells are damaged, or subjected to cell death, as a result of reduced blood flow (ischemia) or as a result of reduced oxygen content (hypoxia).

Ischemia and hypoxia may occur in subjects under different situations, such as under conditions of stroke, acute ischemic stroke, transient ischemic attack, acute hemorrhagic stroke, head trauma, brain hemorrhages, cardiac arrest, cerebral edema, hydrocephalus, asphyxia, vaso-occlusive conditions, embolism, thrombosis, thromboembolism, atherosclerosis, prolonged severe hypotension, intrauterine hypoxia, birth hypoxia, cardiac surgery complications or neurosurgery complications

As used herein, the term “treatment” includes the curative treatment or the preventive treatment of brain ischemic or hypoxic injuries.

A curative treatment is used once ischemia or hypoxia is induced in a subject. The treatment is then typically used to reduce neuronal damage or neuronal cell death in subjects suffering from ischemia or hypoxia. The curative treatment also includes health improvement and recovery of patients having experienced pathological conditions resulting in reduced blood flow or in reduced oxygen content, as described above, by limiting neuronal cell death and brain damage in these subjects. The curative treatment also includes the diminution of secondary ischemic injuries such as reperfusion injuries. A curative acute treatment is preferably started as early as possible upon detection of ischemia or hypoxia, more preferably within hours.

The preventive treatment may be used in subjects at risk of suffering from cerebral ischemia or hypoxia, in order to prevent, lessen or retard neurological insults caused by ischemia or hypoxia. The preventive treatment includes a protection of neuronal cells from damage or death in conditions of glucose and oxygen deprivation as well as in conditions of glutamate excitotoxicity. Subjects at risk include subjects susceptible to suffering from, or who have already suffered from, stroke, acute ischemic stroke, transient ischemic attack, acute hemorrhagic stroke, head trauma, brain hemorrhages, cardiac arrest, cerebral edema, hydrocephalus, asphyxia, vaso-occlusive conditions, embolism, thromboembolism, atherosclerosis, or prolonged severe hypotension. It is well known that patients having suffered from many of the above conditions are likely to relapse.

Consequently, the herein-documented protective effect of compound combinations of the invention can be used as an efficient preventive therapy in such patients at risk. The subjects at risk also include subjects who undergo or who will undergo cardiac surgery or neurosurgery. Furthermore, the preventive therapy of the invention can also be used for other patients with elevated risk factors for developing cerebral ischemia or hypoxia, in relation to hypertension, cigarette smoking, physical inactivity, obesity, cardiovascular disease, high levels of total cholesterol, and atrial fibrillation. Pregnant women are also at increased risk during pregnancy and the postpartum period if they develop cardiovascular conditions such as preeclampsia.

The term “combination” designates a treatment wherein at least two drugs are co-administered to a subject to cause a biological effect. In a combined therapy or composition according to this invention, the at least two drugs may be administered together or separately, at the same time or sequentially. Also, the at least two drugs may be administered through different routes and protocols. As a result, although they may be formulated together, the drugs of a combination may also be formulated separately.

The term “salt” refers to a pharmaceutically acceptable and relatively non-toxic, inorganic or organic acid addition salt of a compound of the present invention. Pharmaceutical salt formation typically consists of pairing an acidic, basic or zwitterionic drug molecule with a counter ion to create a salt version of the drug. A wide variety of chemical species can be used in neutralization reactions. Though most salts of a given active principle are bioequivalents, some may have, among others, increased solubility or bioavailability properties. Salt selection is now a common standard operation in the process of drug development as taught by H. Stahl and C. G. Wermuth in their handbook (24). Pharmaceutically acceptable salts of the invention thus include those obtained by reacting the main compound, functioning as a base, with an inorganic or organic acid to form a salt, for example, salts of acetic acid, nitric acid, tartaric acid, hydrochloric acid, sulfuric acid, phosphoric acid, methane sulfonic acid, camphor sulfonic acid, oxalic acid, maleic acid, succinic acid or citric acid. Pharmaceutically acceptable salts of the invention also include those in which the main compound functions as an acid and is reacted with an appropriate base to form, e.g., sodium, potassium, calcium, magnesium, ammonium, or choline salts.

The term “prodrug” as used herein refers to any derivative (or precursor) of a compound which, when administered to a biological system (e.g., a human organism), generates said compound as a result of, e.g., spontaneous chemical reaction(s), enzyme-catalyzed chemical reaction(s), and/or metabolic chemical reaction(s). Prodrugs are usually inactive or less active than the resulting drug and can be used, for example, to improve the physicochemical properties of the drug, to target the drug to a specific tissue, to improve the pharmacokinetic and pharmacodynamic properties of the drug and/or to reduce undesirable side effects. Specific technical guidance for the selection of suitable prodrugs is general common knowledge. Furthermore, the preparation of prodrugs may be performed by conventional methods known by those skilled in the art. Methods which can be used to synthesize prodrugs are described in numerous reviews on the subject.

The term “metabolite” of a drug as used herein refers to a molecule which results from the (biochemical) modification(s) or processing of said drug after administration to an organism, usually through specialized enzymatic systems, and which displays or retains a biological activity of the drug. Metabolites have been disclosed as being responsible for much of the therapeutic action of the parent drug. In a specific embodiment, a “metabolite” as used herein designates a modified or processed drug that retains at least part of the activity of the parent drug, preferably that has a protective activity against ischemia- or hypoxia-induced neuronal cell death.

The term “derivative” of a compound includes any molecule that is functionally and/or structurally related to said compound, such as an acid, amide, ester, ether, acetylated variant, hydroxylated variant, or an alkylated (C1-C6) variant of such a compound. The term derivative also includes structurally related compounds having lost one or more substituent as listed above.

Similar compounds along with their index of similarity to a parent molecule can be found in numerous databases such as PubChem (see the Worldwide Website for PubChem) or DrugBank (see WorldWide Website fro DrugBank). In a more preferred embodiment, derivatives should have a Tanimoto similarity index greater than 0.4, preferably greater than 0.5, more preferably greater than 0.6, even more preferably greater than 0.7 with a parent drug. The Tanimoto similarity index is widely used to measure the degree of structural similarity between two molecules. Tanimoto similarity index can be computed by software such as the Small Molecule Subgraph Detector available online (see Worldwide Website for Molecule Subgraph Detector). Preferred derivatives should be both structurally and functionally related to a parent compound, i.e., they should also retain at least part of the activity of the parent drug, more preferably they should have a protective activity against ischemia- or hypoxia-induced neuronal cell death.

In a preferred embodiment, the designation of a compound or drug is meant to designate the compound per se, as well as any pharmaceutically acceptable salt, prodrug, metabolite, derivative, hydrate, isomer, or racemate thereof, of any chemical purity.

In an alternate embodiment, a sustained-release formulation of the compound is used.

As discussed above, the invention relates to particular drug combinations which allow an effective protection of neuronal cells from ischemia or hypoxia in individuals in need thereof. More specifically, the invention discloses compositions comprising various combinations of Valproate, Ketamine, Tacrolimus, Clevidipine and Joro Spider Toxin which provide significant protection of neuronal cells against ischemia or hypoxia.

As disclosed in the examples, the inventors have found that combinatorial treatments comprising at least: Valproate, Ketamine, Tacrolimus, Clevidipine and Joro Spider Toxin, have a strong protective effect on neuronal cells in conditions of glucose and oxygen deprivation in a model of ischemia/hypoxia. The results further show that these combinatorial treatments also effectively protect neurons against glutamate toxicity. The results show an unexpected synergistic effect of the combined drugs.

The present invention therefore provides a novel treatment of ischemia or hypoxia, in particular a novel treatment for protecting neuronal cells from ischemia or hypoxia, with a composition comprising any one of the following combinations: Valproate, Ketamine, Tacrolimus, Clevidipine and Joro Spider Toxin.

Thus, one embodiment of the invention is the use of any one the above combinations of drugs, or their pharmaceutically acceptable salts, enantiomers, racemates, prodrugs, derivatives or metabolites, for protecting neuronal cells from ischemia or hypoxia in a subject in need thereof.

In a particular embodiment, the invention relates to the use of the above-mentioned combinations of drugs, or their pharmaceutically acceptable salts, enantiomers, racemates, prodrugs, derivatives or metabolites, for preventing, lessening or retarding neuronal cell death in a subject having a brain ischemia or hypoxia.

Another embodiment of this invention relates to the use of any of the above combinations of drugs, or their pharmaceutically acceptable salts, enantiomers, racemates, prodrugs, derivatives or metabolites, for protecting neuronal cells from death in a subject at risk of having brain ischemia or hypoxia. As discussed before, subjects at risk include subjects having experienced before a brain ischemia or hypoxia, as well as subjects experiencing or susceptible to experiencing a condition or disease which can lead to cerebral ischemia or hypoxia.

The invention is particularly suited to treat brain ischemia or hypoxia in a subject suffering from, or having suffered from, or at risk of suffering from, stroke, shock, transient ischemic attack, cerebral edema, brain hemorrhages, hydrocephalus, trauma, intrauterine or birth hypoxia, embolus, atherosclerosis, or myocardial infarction.

The combination treatments of the invention are also effective and suitable to protect neuronal cells from ischemia or hypoxia in a subject undergoing cardiac surgery or neurosurgery. The treatment may be applied prior to, during, and/or after the surgery.

A preferred embodiment of the invention is the use of one of the above drug combinations for treating brain ischemia or hypoxia, more particularly for protecting neuronal cells in an individual having suffered from, suffering from, or at risk of suffering from, stroke.

It is well known that transient ischemic attacks (TIA) are a harbinger of ischemic stroke, with half of strokes occurring within the first 2 days, and a stroke risk within 90 days over 50 times the normal risk.

Hence, another preferred embodiment of the invention is the use of one of the above combinations for treating brain ischemia or hypoxia, more particularly for protecting neuronal cells from ischemia or hypoxia in an individual having suffered from a transient ischemic attack. This embodiment therefore encompasses the use of the combinations of the invention in conjunction with other drugs or devices aiming at restoring blood or oxygen supply to the brain.

In a further particular embodiment, the invention also relates to the use of one of the above combinations for treating brain ischemia or hypoxia, more particularly for protecting neuronal cells from reperfusion injury in an individual in need thereof.

Another aspect of the invention relates to the use of any of the above combinations of drugs in the manufacture of a medicament for treating brain ischemia or hypoxia, more particularly for protecting neuronal cells from ischemia or hypoxia, in a subject.

In a preferred embodiment, the invention relates to the use of any of these compositions in the manufacture of a medicament for preventing, lessening or retarding neuronal death resulting from an ischemic or hypoxic condition or disease. In another preferred embodiment, the invention relates to the use of any of the compositions of the invention in the manufacture of a medicament for preventing, lessening, or retarding neuronal cell death in subjects suffering from, or at risk of suffering from, stroke.

As indicated previously, in a combination therapy of this invention, the compounds or drugs may be formulated together or separately, and administered together, separately or sequentially.

A further object of the invention is a method of treating brain ischemia or hypoxia in a subject in need thereof, the method comprising simultaneously, separately or sequentially administering to said subject an effective amount of a drug combination as disclosed above.

A further object of the invention is a method of treating, preventing, lessening or retarding ischemia- or hypoxia-induced neuronal cell death, the method comprising simultaneously, separately or sequentially administering to a subject in need thereof an effective amount of a drug combination as disclosed above.

In a preferred embodiment, the invention relates to a method of treating, preventing, lessening or retarding ischemia- or hypoxia-induced neuronal cell death in a subject in need thereof, comprising administering simultaneously, separately or sequentially to the subject an effective amount of: Valproate, Ketamine, Tacrolimus, Clevidipine and Joro Spider Toxin.

For use in the present invention, the drugs or compounds are usually mixed with pharmaceutically acceptable excipients or carriers.

As exemplified, the compound combinations of the invention strongly protect neuronal cells from oxygen and glucose deprivation as well as against glutamate toxicity. The combination treatment is effective at low doses, where the compounds alone have no substantial effect. The invention therefore provides an effective treatment, with reduced risks of side effects, notably during a long-term treatment.

The invention also relates to a pharmaceutical composition per se comprising: Valproate, Ketamine, Tacrolimus, Clevidipine and Joro Spider Toxin, or their pharmaceutically acceptable salts, enantiomers, racemates, prodrugs, derivatives or metabolites. The pharmaceutical compositions of the invention typically further comprise one or several pharmaceutically acceptable excipient(s) or carrier(s).

A further object of this invention is a method of preparing a pharmaceutical composition, the method comprising mixing an appropriate quantity of the above compounds in an appropriate excipient or carrier.

The compositions and methods of the invention are very effective and can be used alone, with no further active agent or treatment. Alternatively, although very effective, depending on the subject or specific condition, the combinations or compositions of the invention may comprise additional active compounds and/or may be used in conjunction or association or combination with additional drugs or treatments. Other additional therapies used in conjunction with drug(s) or drug(s) combination(s) according to the present invention may comprise one or more drug(s) or vascular device(s) currently used to reperfuse ischemic or hypoxic areas, drug(s) which aim to prevent neuronal damages or even one or more drug(s) currently evaluated in the frame of clinical trials.

Therapy according to the invention may be provided at home, the doctor's office, a clinic, a hospital's outpatient department, or a hospital, so that the doctor can observe the therapeutic effects closely and make any adjustments needed.

The duration of the therapy depends on the stage of the disease being treated, age and condition of the patient, and how the patient responds to the treatment. The dosage, frequency and mode of administration of each component of the combination can be controlled independently. For example, one drug may be administered orally while the second drug may be administered intramuscularly. Combination therapy may be given in on-and-off cycles that include rest periods so that the patient's body has a chance to recover from any unforeseen side effects. The drugs may also be formulated together such that one administration delivers all drugs.

The administration of each drug of the combination may be by any suitable means that results in a concentration of the drug that, combined with the other component, is able to prevent, retard, or impede neuronal cell death and neurological damage stemming from cerebral ischemia or hypoxia.

While it is possible for the drugs in the combination to be administered as the pure chemicals it is preferable to present them as a pharmaceutical composition, also referred to in this context as a pharmaceutical formulation. Possible compositions include those suitable for oral, rectal, topical (including transdermal, buccal and sublingual), or parenteral (including subcutaneous, intramuscular, and intravenous administration.

More commonly these pharmaceutical formulations are prescribed to the patient in “patient packs” containing a number of dosing units or other means for administration of metered unit doses for use during a distinct treatment period in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions. Thus, the invention further includes a pharmaceutical formulation, as described herein, in combination with packaging material suitable for said formulation. In such a patient pack the intended use of a formulation for the combination treatment can be inferred by instructions, facilities, provisions, adaptations and/or other means to help use the formulation most suitably for the treatment. Such measures make a patient pack specifically suitable for and adapted to use for treatment with the combination of the present invention.

The drug may be contained, in any appropriate amount, in any suitable carrier substance. The drug may be present in an amount of up to 99% by weight of the total weight of the composition. The composition may be provided in a dosage form that is suitable for the oral, parenteral (e.g., intravenous, intramuscular), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols.

The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active drug substantially immediately upon administration or at any predetermined time or time period after administration.

The controlled release formulations include (i) formulations that create a substantially constant concentration of the drug within the body over an extended period of time; (ii) formulations that after a predetermined lag time create a substantially constant concentration of the drug within the body over an extended period of time; (iii) formulations that sustain drug action during a predetermined time period by maintaining a relatively, constant, effective drug level in the body with concomitant minimization of undesirable side effects associated with fluctuations in the plasma level of the active drug substance; (iv) formulations that localize drug action by, e.g., spatial placement of a controlled release composition adjacent to or in the diseased tissue or organ; and (v) formulations that target drug action by using carriers or chemical derivatives to deliver the drug to a particular target cell type.

Administration of drugs in the form of a controlled release formulation is especially preferred in cases in which the drug has (i) a narrow therapeutic index (i.e., the difference between the plasma concentration leading to harmful side effects or toxic reactions and the plasma concentration leading to a therapeutic effect is small; in general, the therapeutic index, TI, is defined as the ratio of median lethal dose (LD₅₀) to median effective dose (ED₅₀)); (ii) a narrow absorption window in the gastrointestinal tract; or (iii) a very short biological half-life so that frequent dosing during a day is required in order to sustain the plasma level at a therapeutic level.

Any of a number of strategies can be pursued in order to obtain controlled release in which the rate of release outweighs the rate of metabolism of the drug in question. Controlled release may be obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner (single or multiple unit tablet or capsule compositions, oil solutions, suspensions, emulsions, microcapsules, microspheres, nanoparticles, patches, and liposomes).

### Solid Dosage Forms for Oral Use

Formulations for oral use include tablets containing the composition of the invention in a mixture with non-toxic pharmaceutically acceptable excipients. These excipients may be, for example, inert diluents or fillers (e.g., sucrose, microcrystalline cellulose, starches including potato starch, calcium carbonate, sodium chloride, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., acacia, alginic acid, sodium alginate, gelatin, starch, pregelatinized starch, microcrystalline cellulose, carboxymethylcellulose sodium, methylcellulose, hydroxypropyl methylcellulose, ethyl cellulose, polyvinylpyrrolidone, or polyethylene glycol); and lubricating agents, glidants, and anti-adhesives/disintegrants (e.g., stearic acid, silicas, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby provide a sustained action over a longer period. The coating may be adapted to release the active drug substance in a predetermined pattern (e.g., in order to achieve a controlled release formulation) or it may be adapted not to release the active drug substance until after passage of the stomach (enteric coating). The coating may be a sugar coating, a film coating (e.g., based on hydroxypropyl methylcellulose, methylcellulose, methyl hydroxyethyl cellulose, hydroxypropylcellulose, carboxymethylcellulose, acrylate copolymers, polyethylene glycol and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). A time delay material such as glyceryl monostearate or glyceryl distearate may be employed.

The solid tablet compositions may include a coating adapted to protect the composition from unwanted chemical changes (e.g., chemical degradation prior to the release of the active drug substance). The coating may be applied on the solid dosage form in a similar manner as that described in the Encyclopedia of Pharmaceutical Technology.

Drugs may be mixed together in the tablet, or may be partitioned. For example, a first drug is contained on the inside of the tablet, and a second drug is on the outside, such that a substantial portion of the second drug is released prior to the release of the first drug.

Formulations for oral use may also be presented as chewable tablets, as hard gelatin capsules wherein the active ingredient is mixed with an inert solid diluent (e.g., potato starch, microcrystalline cellulose, calcium carbonate, calcium phosphate or kaolin), or as soft gelatin capsules wherein the active ingredient is mixed with water or an oil medium, for example, liquid paraffin or olive oil. Powders and granulates may be prepared using the ingredients mentioned above under tablets and capsules in a conventional manner.

Controlled release compositions for oral use may, e.g., be constructed to release the active drug by controlling the dissolution and/or the diffusion of the active drug substance.

Dissolution or diffusion controlled release can be achieved by appropriate coating of a tablet, capsule, pellet, or granulate formulation of drugs, or by incorporating the drug into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogel, 1,3 butylene glycol, ethylene glycol methacrylate, and/or polyethylene glycol. In a controlled release matrix formulation, the matrix material may also include, e.g., hydrated methylcellulose, carnauba wax, stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more of the drugs of the claimed combinations may also be in the form of a buoyant tablet or capsule (i.e., a tablet or capsule that, upon oral administration, floats on top of the gastric content for a certain period of time). A buoyant tablet formulation of the drug(s) can be prepared by granulating a mixture of the drug(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Liquids for Oral Administration

Powders, dispersible powders, or granules suitable for preparation of an aqueous suspension by addition of water are convenient dosage forms for oral administration. Formulation as a suspension provides the active ingredient in a mixture with a dispersing or wetting agent, suspending agent, and one or more preservatives. Suitable suspending agents are, for example, sodium carboxymethylcellulose, methylcellulose, sodium alginate, and the like.

### Parenteral Compositions

The pharmaceutical composition may also be administered parenterally by injection, infusion or implantation (intravenous, intramuscular, subcutaneous, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation or it may be presented as a dry powder to be reconstituted with water or another suitable vehicle before use. Apart from the active drug(s), the composition may include suitable parenterally acceptable carriers and/or excipients. The active drug(s) may be incorporated into microspheres, microcapsules, nanoparticles, liposomes, or the like for controlled release. The composition may include suspending, solubilizing, stabilizing, pH-adjusting, and/or dispersing agents.

The pharmaceutical compositions according to the invention may be in a form suitable for sterile injection. To prepare such a composition, the suitable active drug(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution. The aqueous formulation may also contain one or more preservatives (e.g., methyl, ethyl or n-propyl p-hydroxybenzoate). In cases where one of the drugs is only sparingly or slightly soluble in water, a dissolution enhancing or solubilizing agent can be added, or the solvent may include 10-60% w/w of propylene glycol or the like.

Controlled release parenteral compositions may be in form of aqueous suspensions, microspheres, microcapsules, magnetic microspheres, oil solutions, oil suspensions, or emulsions. Alternatively, the active drug(s) may be incorporated in biocompatible carriers, liposomes, nanoparticles, implants, or infusion devices. Materials for use in the preparation of microspheres and/or microcapsules are, e.g., biodegradable/bioerodable polymers such as polygalactin, poly-(isobutyl cyanoacrylate), or poly(2-hydroxyethyl-L-glutamnine). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(glycolic acid) or poly(orthoesters)).

### Alternative Routes

Although less preferred and less convenient, other administration routes, and therefore other formulations, may be contemplated. In this regard, for rectal application, suitable dosage forms for a composition include suppositories (emulsion or suspension type) and rectal gelatin capsules (solutions or suspensions). In a typical suppository formulation, the active drug(s) are combined with an appropriate pharmaceutically acceptable suppository base such as cocoa butter, esterified fatty acids, glycerinated gelatin, and various water-soluble or dispersible bases like polyethylene glycol. Various additives, enhancers, or surfactants may be incorporated.

The pharmaceutical compositions may also be administered topically on the skin for percutaneous absorption in dosage forms or formulations containing conventionally non-toxic pharmaceutically acceptable carriers and excipients including microspheres and liposomes. The formulations include creams, ointments, lotions, liniments, gels, hydrogels, solutions, suspensions, sticks, sprays, pastes, plasters, and other kinds of transdermal drug delivery systems. The pharmaceutically acceptable carriers or excipients may include emulsifying agents, antioxidants, buffering agents, preservatives, humectants, penetration enhancers, chelating agents, gel-forming agents, ointment bases, perfumes, and skin protective agents.

The preservatives, humectants, and penetration enhancers may be parabens, such as methyl or propyl p-hydroxybenzoate, benzalkonium chloride, glycerin, propylene glycol, urea, etc.

The pharmaceutical compositions described above for topical administration on the skin may also be used in connection with topical administration onto or close to the part of the body that is to be treated. The compositions may be adapted for direct application or for application by means of special drug delivery devices such as dressings or alternatively plasters, pads, sponges, strips, or other forms of suitable flexible material.

### Dosages and Duration of the Treatment

It will be appreciated that the drugs of the combination may be administered concomitantly, either in the same or different pharmaceutical formulation.

Therapeutically effective amounts of the drugs in a combination of this invention include, e.g., amounts effective for reducing neuronal cell death, thereby halting or slowing the progression of the neurological damage or symptoms thereof once they manifest clinically.

Although the active drugs of the present invention may be administered in divided doses, for example two or three times daily, a single daily dose of each drug in the combination is preferred, with a single daily dose of all drugs in a single pharmaceutical composition (unit dosage form) being most preferred.

Administration can be one to three times daily for several days; longer time courses are probably safe but would raise a patient's risk of adverse effects, mainly hepatotoxicity and opportunistic infections associated with long term use of Tacrolimus. Though the treatment might be limited to hours or several days after the onset of the ischemic or hypoxic event, chronic or at least periodically repeated long-term administration might be also indicated for patients with a high risk of experiencing cerebral ischemia or hypoxia.

The term “unit dosage form” refers to physically discrete units (such as capsules, tablets, or loaded syringe cylinders) suitable as unitary dosages for human subjects, each unit containing a predetermined quantity of active material or materials calculated to produce the desired therapeutic effect, in association with the required pharmaceutical carrier.

The amount of each drug in a preferred unit dosage composition depends upon several factors including the administration method, the body weight and the age of the patient, the stage of the ischemic or hypoxic event, and the risk of potential side effects considering the general health status of the person to be treated. Additionally, pharmacogenomic (the effect of genotype on the pharmacokinetic or pharmacodynamic profile of a therapy) information about a particular patient may affect the dosage used.

Tacrolimus, at dose concentration of 0.1 mg/kg is neuroprotective. This works out to 7-10 mg for the average adult patient dosed by body weight. Lower doses of Tacrolimus are not neuroprotective—at least not when the drug is used as monotherapy. In the case of CVA, it makes sense to start at the high end of the dosage range and decrease the dose if adverse effects occur. The main advantage of an IV drip is that it can be stopped at any time. In contrast, an oral capsule or rectal suppository cannot be retrieved.

Specific examples of dosages of drugs for use in the invention are provided below: Valproate, between 1000 and 3000 mg per day, Ketamine, between 400 and 800 mg per day, Tacrolimus, from 10 to 30 mg per day, Clevidipine from about 5 to 30 mg per day, and Joro Spider Toxin from about 1 to 2 mcg per day. It should be noted that maximum safe daily dose of JSTX has not been established for humans.

It will be understood that the amount of the drug actually administered shall be determined and adapted by a physician, in light of the relevant circumstances including the condition or conditions to be treated, the exact composition to be administered, the age, weight, and response of the individual patient, the severity of the patient's symptoms, and the chosen route of administration. Therefore, the above dosage ranges are intended to provide general guidance and support for the teachings herein, but are not intended to limit the scope of the invention.

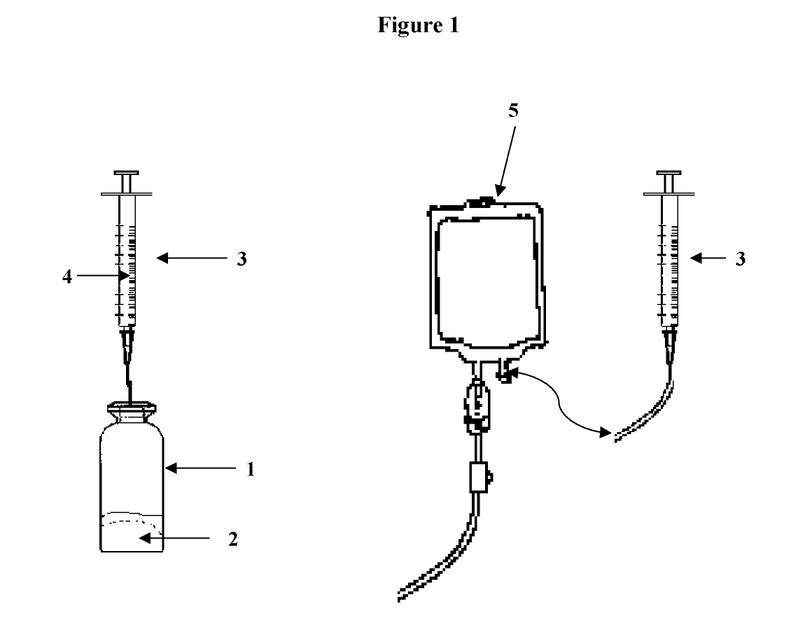

Referring to FIG. 1, one of the embodiments of the invention shows a sterile glass vial 1, containing a dry powder 2 to which there is added a normal saline solution or other diluent 4 inside syringe 3. The dissolved powders are then are then added into IV bag 5 via syringe 3.

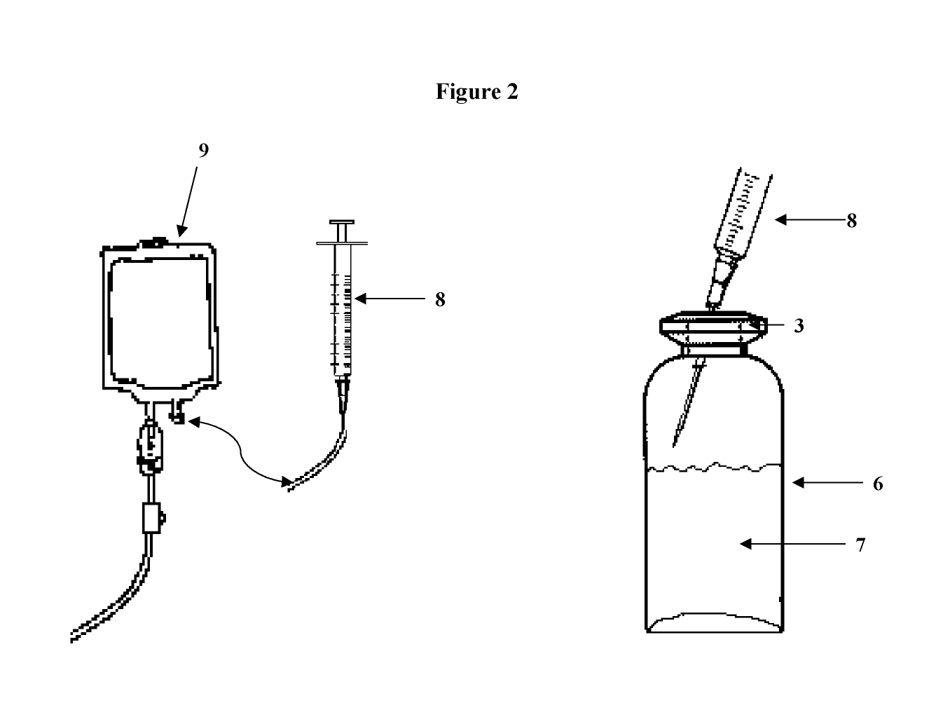

In another embodiment shown in FIG. 2, there is shown a sterile glass vial 6 having the active ingredients as concentrated liquids 7 which are aspirated with syringe 8 and then introduced into IV bag 9 via syringe 8.

Further aspects and advantages of the invention will be disclosed in the following experimental section, which serves to illustrate the invention at the in vitro level.

### EXAMPLES

### Example 1

### Protective Effect of Combination Treatments of the Invention Against Ischemia/Hypoxia-Induced Neuronal Cell Death

### Methods

### Rat Neuronal Cortical Cell Preparation

Rat cortical neurons are cultured as described by Singer et al. (27). Briefly pregnant female rats of 15 days gestation are killed by cervical dislocation (Rats Wistar) and the fetuses are removed from the uterus. The cortex is removed and placed in ice-cold medium of Leibovitz (L15) containing 2% of Penicillin 10,000 U/ml and Streptomycin 10 mg/mL and 1% of bovine serum albumin (BSA). Cortices are dissociated by trypsin for 20 min at 37° C. (0.05%). The reaction is stopped by the addition of Dulbecco's modified Eagle's medium (DMEM) containing DNase 1 grade II and 10% of fetal calf serum (FCS). Cells are then mechanically dissociated by 3 serial passages through a 10 mL pipette and centrifuged at 515 g for 10 min at +4° C. The supernatant is discarded and the pellet of cells is resuspended in a defined culture medium consisting of Neurobasal supplemented with B27 (2%), L-glutamine (0.2 mM), 2% of PS solution and 10 ng/mL of BDNF. Viable cells are counted in a Neubauer cytometer using the trypan blue exclusion test. The cells are seeded at a density of 30,000 cells/well in 96-well plates (wells were pre-coated with poly-L-lysine (10 mcg/mL)) and are cultured at 37° C. in humidified air (95%) with 5% CO₂.

### Oxygen and Glucose Deprivation Assays (In Vitro Model of Ischemia)

The neuroprotective effect of compounds is assessed by quantification of the neurite network (Neurofilament immunostaining (NF)) using MAP2 antibody. Riluzole, a neuroprotective drug, (RILUTECK, 5.mu.M) is used as positive control.

After 10 days of neuron culture, candidate drugs are dissolved in culture medium (+0.1% DMSO) and then pre-incubated with neurons for 1 hour before the oxygen and glucose deprivation. One hour after candidate drug incubation, the medium is removed and fresh medium without glucose is added. This medium is composed of DMEM without glucose (Invitrogen) supplemented with 2% B27, 0.2 mM L-glutamine, 1% PS solution, and 10 ng/mL of BDNF. The cells are transferred into an anaerobic incubator with 95% N₂ and 5% CO₂ at 37° C. After 2 hours, 25 mM of D-Glucose will be added to the culture medium and cells are transferred to a classic incubator with 95% air/5% CO₂ at 37° C. After 24 hours of oxygen glucose reperfusion, cells are fixed by a cold solution of alcohol/acetic acid for 5 minutes.

After permeabilization with saponin (Sigma), cells are blocked for 2 hours with PBS containing 10% goat serum, then the cells are incubated with mouse monoclonal primary antibody against MAP2 (MAP2, Sigma). These antibodies are revealed with Alexa Fluor 488 goat anti-mouse IgG (Molecular probe).

Nuclei of cells are labeled by a fluorescent marker (Hoechst solution, SIGMA). Six wells per condition are used to assess neuronal survival in 3 different cultures. For each condition 2×10 pictures per well are taken and analyzed using InCell Analyzer™ 1000 (GE Healthcare) with 20.times. magnification.

### Results

All of the claimed drug combinations give a protective effect against ischemia/hypoxia-induced cell death for cortical neuronal cells.

These results therefore demonstrate a potent and synergistic effect of the claimed combination therapies on brain ischemia and hypoxia.

### Example 2

### Protective Effect of Combination Treatments of the Invention Against Glutamate Toxicity

Glutamate toxicity is often described in the literature as being a part of the ischemic cascade that leads to neuronal cell death. Combination therapies have been tested for their protective effect against glutamate toxicity in vitro.

### Methods

Rat neuronal cortical cells are prepared as in Example 1 section.

### Glutamate Toxicity Assays

The neuroprotective effect of compounds is assessed by quantification of the neurite network (Neurofilament immunohistochemistry (NF)) which specifically reveals the glutamatergic neurons.

After 12 days of neuron culture, drugs of the candidate combinations are dissolved in culture medium (+0.1% DMSO). Candidate combinations are then pre-incubated with neurons for 1 hour before the glutamate injury. One hour after incubation, glutamate is added for 20 min, to a final concentration of 40 microM (or use the notation μM), in the presence of candidate combinations, in order to avoid further drug dilutions. At the end of the incubation, the medium is changed with medium with candidate combination but without glutamate. The culture is fixed 24 hours after glutamate injury. MK801 (20.mu.M) is used as a positive control.

After permeabilization with saponin (Sigma), cells are blocked for 2 h with PBS containing 10% goat serum, then the cells are incubated with mouse monoclonal primary antibody against Neurofilament antibody (NF, Sigma). This antibody is revealed with Alexa Fluor 488 goat anti-mouse IgG (Molecular probe).

Nuclei of cells are labeled by a fluorescent marker (Hoechst solution, SIGMA), and the neurite network is quantified. Six wells per condition are used to assess neuronal survival in 3 different cultures.

### Results

All of the claimed drug combinations provide a protective effect against glutamate toxicity for cortical neuronal cells.

The present invention covers dry powder and liquid APIs respectively. They are intended to be administered intravenously following dilution in an IV bag containing normal saline (NS) or any other suitable diluent. The embodiments of the invention may be administered in an ambulance, urgent care center or hospital once IV access has been established (peripheral IV, PICC line, or central venous catheter). Intravenous delivery is the preferred route of administration for the drug combinations of the invention.

In another embodiment, an oral capsule intended to be given to patients experiencing signs/symptoms of TIA or stroke who are conscious and capable of swallowing but have poor IV access.

Similarly a rectal suppository. The PR route is reserved for patients who are found unconscious or are too obtunded to swallow and who have poor IV access.

### Example 3

A pressurized sterile glass vial containing the following active pharmaceutical ingredients (API) in lyophilized/dry powder form for intravenous reconstitution.

### Valproate 500 mg

### Ketamine 400 mg

### Tacrolimus 10 mg

### Clevidipine 5 mg

### Joro Spider Toxin 1 mcg

A diluent (normal saline) would be used to dissolve the API in a volume of approximately 10 mL. The dissolved API would then be added to a larger IV bag containing 500-1000 mL of normal saline or other IV fluid.

Shelf life of dry powder API prior to reconstitution: 12-24 months at room temperature

### Example 4

A sterile glass vial containing 5 API in concentrated liquid form (30 mL or 1 oz vial):

Valproate/Depacon 100 mg/mL×5 mL
Ketamine 100 mg/mL×4 mL
Tacrolimus 5 mg/5 mL×2
Clevidipine 0.5 mg/mL×10 mL

### JSTX 1 mcg

The liquid API (˜25 mL) would be injected into a 500 mL-1000 mL IV bag as in Example 3.
Anticipated shelf life: 90-120 days at room temperature

### Example 5

An oral capsule for Transient Ischemic Attack/stroke patients with difficult IV access who remain conscious and are capable of swallowing. Capsule may be disassembled and its contents dissolved in several ounces of water or cola for patients unable to swallow a size zero or size one capsule:

### Valproate 500 mg

### Ketamine HCl 400 mg

### Tacrolimus 10 mg

### Clevidipine 5 mg

### JSTX 1 mcg

Anticipated shelf life: 18-24 months at room temperature

### Example 6

A rectal suppository containing the quantities of API listed above in Example 5. The PR route is reserved for patients who are found unconscious; are too obtunded to swallow and who have poor IV access. A suppository can be administered at home, in an ambulance or at a hospital prior to placement of an IV/PICC line/central venous catheter.

Anticipated shelf life: 12-18 months at room temperature

### Example 7

A pressurized sterile glass vial containing the following active pharmaceutical ingredients (API) in lyophilized/dry powder form for intravenous reconstitution.

### Valproate 500 mg

### Ketamine 400 mg

### Tacrolimus 10 mg

### Nicardipine 30 mg

### Joro Spider Toxin 1 mcg

A diluent (normal saline) would be used to dissolve the API in a volume of approximately 10 mL. The dissolved API would then be added to a larger IV bag containing 500-1000 mL of normal saline or other IV fluid.

### Example 8

A sterile glass vial containing 5 API in concentrated liquid form (30 mL or 1 oz vial):

Valproate/Depacon 100 mg/mL×5 mL
Ketamine 100 mg/mL×4 mL
Tacrolimus 5 mg/5 mL×2
Nicardipine (Cardene) premix 30 mg/mL

### JSTX 1 mcg

The liquid API (˜25 mL) would be injected into a 500 mL-1000 mL IV bag as in #1.

### Example 9

An oral capsule for Transient Ischemic Attack/stroke patients with difficult IV access who remain conscious and are capable of swallowing. Capsule may be disassembled and its contents dissolved in several ounces of water or cola for patients unable to swallow a size zero or size one capsule:

### Valproate 500 mg

### Ketamine HCl 400 mg

### Tacrolimus 10 mg

### Nifedipine 30 mg

### JSTX 1 mcg

### Example 10

A pressurized sterile glass vial containing the following active pharmaceutical ingredients (API) in lyophilized/dry powder form for intravenous reconstitution.

### Valproate 500 mg

### Ketamine 400 mg

### Tacrolimus 10 mg

A diluent (normal saline) would be used to dissolve the API in a volume of approximately 10 mL. The dissolved API would then be added to a larger IV bag containing 500-1000 mL of normal saline or other IV fluid.

### Example 11

A pressurized sterile glass vial containing the following active pharmaceutical ingredients (API) in lyophilized/dry powder form for intravenous reconstitution.

### Valproate 500 mg

### Ketamine 400 mg

A diluent (normal saline) would be used to dissolve the API in a volume of approximately 10 mL. The dissolved API would then be added to a larger IV bag containing 500-1000 mL of normal saline or other IV fluid.

### Example 12

A pressurized sterile glass vial containing the following active pharmaceutical ingredients (API) in lyophilized/dry powder form for intravenous reconstitution.

### Valproate 500 mg

### Ketamine 400 mg

### Clevidipine 5 mg

A diluent (normal saline) would be used to dissolve the API in a volume of approximately 10 mL. The dissolved API would then be added to a larger IV bag containing 500-1000 mL of normal saline or other IV fluid.

### Example 13

A pressurized sterile glass vial containing the following active pharmaceutical ingredients (API) in lyophilized/dry powder form for intravenous reconstitution.

### Valproate 500 mg

### Clevidipine 5 mg

### Tacrolimus 10 mg

A diluent (normal saline) would be used to dissolve the API in a volume of approximately 10 mL. The dissolved API would then be added to a larger IV bag containing 500-1000 mL of normal saline or other IV fluid.

### Example 14

A pressurized sterile glass vial containing the following active pharmaceutical ingredients (API) in lyophilized/dry powder form for intravenous reconstitution.

### Valproate 500 mg

### Ketamine 400 mg

### Tacrolimus 10 mg

### Clevidipine 5 mg

A diluent (normal saline) would be used to dissolve the API in a volume of approximately 10 mL. The dissolved API would then be added to a larger IV bag containing 500-1000 mL of normal saline or other IV fluid.

All patents, patent applications and publications cited in this application including all cited references in those patents, applications and publications, are hereby incorporated by reference in their entirety for all purposes to the same extent as if each individual patent, patent application or publication were so individually denoted.

While the many embodiments of the invention have been disclosed above and include presently preferred embodiments, many other embodiments and variations are possible within the scope of the present disclosure and in the appended claims that follow. Accordingly, the details of the preferred embodiments and examples provided are not to be construed as limiting. It is to be understood that the terms used herein are merely descriptive rather than limiting and that various changes, numerous equivalents may be made without departing from the spirit or scope of the claimed invention.

What is claimed is:

1. A method for the treatment of a brain ischemic or hypoxic injury in a subject in need thereof, comprising administering to said subject a combination of effective amounts of at least two compounds selected from the group consisting of: Valproate, Ketamine, Tacrolimus, Clevidipine, Nicardipine and Joro Spider Toxin, or their pharmaceutically acceptable salts, enantiomers, racemates, prodrugs, derivatives or metabolites and one or several pharmaceutically acceptable excipient(s) or carrier(s).

2. The method according to claim 1, wherein said ischemic injury is a focal ischemic injury or a global ischemic injury.

3. The method according to claim 1, wherein said subject is suffering from or is susceptible to suffer from stroke, acute ischemic stroke, transient ischemic attack, acute hemorrhagic stroke, head trauma, brain hemorrhages, cardiac arrest, cerebral edema, hydrocephalus, asphyxia, thrombosis, embolism, thromboembolism, atherosclerosis, prolonged severe hypotension, intrauterine hypoxia, birth hypoxia, cardiac surgery complications or neurosurgery complications.

4. The method according to claim 1, to reduce, lessen or retard neuronal damage or neuronal cell death in a subject having ischemia or hypoxia.

5. The method according to claim 1, to protect a subject from brain ischemia or hypoxia.

6. The method according to claim 1, for treating brain ischemia or hypoxia, or for protecting neuronal cells, in a subject having suffered from, suffering from, or at risk to suffer from stroke.

7. The method according to claim 1, for protecting brain neuronal cells from ischemia or hypoxia, in a subject undergoing cardiac or neurosurgery.

8. The method according to claim 1, for treating brain ischemia or hypoxia, or for protecting neuronal cells, in a subject having suffered from a transient ischemic attack.

9. The method according to claim 1, for treating brain ischemia or hypoxia, or for protecting neuronal cells, in a subject at risk to suffer from reperfusion injury.

10. The method according to claim 1, wherein the compounds are admixed with a pharmaceutically acceptable carrier or excipient.

11. The method according to claim 1, wherein the compounds are formulated or administered together, separately or sequentially.

12. The method according to claim 1, wherein said compounds are administered repeatedly to the subject.

13. A pharmaceutical composition for the treatment of a brain ischemic or hypoxic injury comprising a mixture of effective amounts of at least 2 compounds selected from the group consisting of Valproate, Ketamine, Tacrolimus, Clevidipine, Nicardipine and Joro Spider Toxin, or their pharmaceutically acceptable salts, enantiomers, racemates, prodrugs, derivatives or metabolites. and one or several pharmaceutically acceptable excipient(s) or carrier(s).

14. A method for treating cerebral stroke, heart stroke, a neurodegenerative disease, brain trauma, or nervous system trauma in a patient, comprising administering the product of claim 13 to the patient.

15. A method for the treatment of a brain ischemic or hypoxic injury in a subject in need thereof, comprising administering to said subject a combination of effective amounts of compounds selected from the group consisting of: Valproate, Ketamine, Tacrolimus, Clevidipine, and optionally Joro Spider Toxin, or their pharmaceutically acceptable salts, enantiomers, racemates, prodrugs, derivatives or metabolites and one or several pharmaceutically acceptable excipient(s) or carrier(s).

16. The method according to claim 15, wherein said ischemic injury is a focal ischemic injury or a global ischemic injury.

17. The method according to claim 15, wherein said subject is suffering from or is susceptible to suffer from stroke, acute ischemic stroke, transient ischemic attack, acute hemorrhagic stroke, head trauma, brain hemorrhages, cardiac arrest, cerebral edema, hydrocephalus, asphyxia, thrombosis, embolism, thromboembolism, atherosclerosis, prolonged severe hypotension, intrauterine hypoxia, birth hypoxia, cardiac surgery complications or neurosurgery complications.

18. The method according to claim 15, to reduce, lessen or retard neuronal damage or neuronal cell death in a subject having ischemia or hypoxia.

19. The method according to claim 15, to protect a subject from brain ischemia or hypoxia.

20. The method according to claim 15, for treating brain ischemia or hypoxia, or for protecting neuronal cells, in a subject having suffered from, suffering from, or at risk to suffer from stroke.
